(19) United States
(12) Patent Application Publication
(10) Pub. No.: US 2024/0312588 A1
(43) Pub. Date: Sep. 19, 2024

(54) SYSTEM FOR MONITORING THE INTAKE OF SUBSTANCES

(71) Applicant: Aptar Radolfzell GmbH, Radolfzell (DE)

(72) Inventors: Joachim KÖRNER, Uhldingen-Mühlhofen (DE); Julien STORZ-SONTHEIMER, Gottmadingen (DE)

(21) Appl. No.: 18/576,530

(22) PCT Filed: Jul. 4, 2022
(86) PCT No.: PCT/EP2022/068477

§ 371 Date: Jan. 4, 2024

(30) Foreign Application Priority Data
Jul. 5, 2021 (EP) 21183760.4

(51) Int. Cl. G16H 20/13; G16H 40/63

(52) U.S. Cl. CPC G16H 20/13; G16H 40/63

(57) ABSTRACT

A system for monitoring the intake of substances, more particularly pharmaceutical substances or nicotine. The system includes at least two dispensers for dispensing the same substance, the two dispensers being designed to dispense the substance in different dosage forms. The system also includes a monitoring device, which is connected to the at least two dispensers by a data connection. The two dispensers are designed to sense handling of the dispenser associated with delivery of the substance and to transmit to the monitoring device, in response thereto, data regarding the dispensing of the substance. The monitoring device is designed to receive the data regarding the dispensing of the substance from the at least two dispensers and to calculate a total amount of the dispensed substance. The system provides users with an overview of their substance consumption even if they take in the substance different ways.

### AREA OF APPLICATION AND PRIOR ART

The invention relates to a system for monitoring the intake of substances, in particular of pharmaceutical substances or of nicotine.

A large number of routes to introduce active substances into the body are customary, for example in particular the introduction on an oral or nasal route in the form of a liquid, for example in droplet form or in the form of a spray jet, but also in the form of tablets or chewing gum which, following oral consumption, release their active substance in the mouth or in the stomach of the user.

In particular in the area of the introduction of nicotine into the body, it is also customary to use patches for this purpose, which are applied to the skin of the user and supply the substance to the body of the user over a relatively long time.

In the case of many substances and in particular in the case of nicotine, it is desired to be able to estimate the quantity of the substance already introduced into the human body, for example in order to achieve a predefined target value of the introduced substance in a defined time period or in order not to exceed a predefined maximum value.

Thus, in particular in the context of nicotine, for the purpose of limiting the actual consumption or for the purpose of quitting, it is desirable that the user maintains an overview as to how much nicotine he or she has already taken in a defined time period, such as for example on the current day.

From the prior art, dispensers are already known, for example liquid dispensers, which detect the use of the dispenser with electronic means, so that the use of the dispenser and the quantity of liquid taken can be read on the dispenser or on a smartphone connected thereto.

In practice, however, in particular with regard to nicotine, the result is the problem that it is not uncommon for persons to supply these or other substances in a different form. Thus, for example, it is not uncommon for users from time to time to use a patch with the substance in question but, at other times, also, for example, to use chewing gum with the substance. In such a case, there is a high risk that the relevant person loses the overview as to how much substance has already been supplied to their body.

### OBJECT AND SOLUTION

The object of the invention is to provide a possible way by means of which the problem outlined above is countered and persons can maintain an overview of the quantity of the substance ingested.

According to the invention, for this purpose a system and a method are proposed, which are explained jointly below.

The system according to the invention and the method according to the invention can be used to monitor the introduction of different pharmacologically active substances into the human body. This will be explained below with reference to nicotine. However, it can also involve medicaments the introduction of which into the body is medically necessarily required.

The system comprises at least two dispensers which are used to deliver the same substance. The at least two dispensers are provided to deliver the substance in a different application form. The application form can differ as to whether the delivery by the dispensers is used immediately for the introduction into the human body or whether a further application step follows first, such as, for example, the ingestion or application of a piece of chewing gum previously delivered by the dispenser or a patch delivered by the dispenser.

The at least two dispensers are designed to detect action associated with the delivery of a substance. This action can directly be that action which is not only used to deliver the substance from the dispenser but at the same time also the ingestion by the user. This is the case, for example, in a liquid dispenser which is capable of detecting via a sensor the actuation by means of which liquid is applied into the mouth or the nose of the user. However, the action detected can also be that which does not lead directly to the ingestion but takes place chronologically before this. This can be the case, for example, when the dispenser is used to deliver chewing gum or tablets. In this case, the dispenser does not detect the ingestion of this chewing gum or the tablet directly but only the delivery from the dispenser or the opening of the access to a receiving chamber of the dispenser. It is therefore assumed in this case only that the user actually also ingests the chewing gum or the tablet in the intended way following the delivery from the dispenser.

The at least two dispensers, which thus monitor the delivery of the substance, are designed to transmit the information about the delivery to a central monitoring device. The central monitoring device is preferably a device which is a personal device of the user themselves, for example their computer. In particular it can be a smartphone, the functionality of which is extended by a program (app) with a view to the functions described further below.

Via a data link, in particular a WLAN or Bluetooth connection, the at least two dispensers transmit the data about the delivery of the substance to the smartphone or to an otherwise configured central monitoring device. For this purpose, the dispensers preferably each have a radio module. Preferably, the dispensers also have an integrated circuit or a processor for evaluating sensor data and for activating the radio module. Furthermore, it is viewed as advantageous if the dispensers have a dispenser in which they deposit data relating to the action detected, so that the data transmission does not have to take place immediately.

The monitoring device is designed to receive the appropriate data relating to the delivery of the substance from the at least two dispensers.

According to the invention, this data is then incorporated in a calculation of the total quantity of the substance delivered. This means that the monitoring device calculates, by means of its processor, how high the total quantity of the delivered substance from the at least two dispensers is, in particular in a limited time period, such as the current day.

The monitoring device can take into account what quantity of the substance is associated with each delivery from the dispenser. Thus, for example, it is possible to take account of the fact that a single nicotine patch contains a nicotine quantity of, for example, 10 mg, while a nicotine spray delivers only 0.5 mg with each delivery. In order to take this into account, in a possible configuration, the delivery quantity of the substance for each dispenser can be stored in the monitoring device. By contrast, however, it is advantageous if the dispenser itself does not transmit or transmits only the number of delivery operations to the central monitoring device but instead or also transmits the associated delivery quantity. In such a case, the monitoring device does not need to be matched specifically to the respective dispenser.

The total quantity of delivered substance calculated by the monitoring device preferably relates to a defined time period such as, for example, to one day, one week or one month. Accordingly, preferably at regular intervals, this time interval-based total quantity is reset to zero or the delivery operations which are to be taken into account when calculating the total quantity and which are stored in a memory of the monitoring device are limited during the calculation of the total quantity, in particular to that of the current day, the current week or the current month.

The monitoring device, that is to say in particular the smartphone, preferably has an indicating device, in particular in the form of a display. On this indicating device, it is primarily the calculated total quantity of the substance delivered by the dispensers that is indicated, wherein the display of the absolute quantity itself can be provided and, alternatively or additionally, also a percentage value which indicates what proportion of a total quantity or maximum quantity that is envisaged for the relevant time interval has already been reached. Preferably, a graphic representation in the form of a progress bar is also provided, which permits detection of the current status at a glance.

If a maximum quantity or a target quantity for the corresponding time interval, for example the current day, is stored, then in addition the quantity of substance still remaining for the time interval can be determined, starting from the total quantity of substance already delivered in this time interval.

Preferably, provision is made for the monitoring device to be designed to indicate this remaining quantity of substance. In order to make it particularly simple for the user, the monitoring device can be designed to recalculate this remaining quantity of substance such that the number of still available delivery operations is indicated specifically for each dispenser of the system. Thus, for example in the form of numeric values, it is possible to output how many delivery operations and uses of patches or delivery operations of spray would still be available if the total remaining quantity of substance were to be delivered by the respective application method.

The monitoring device is further preferably designed to output visual or acoustic warning messages if the total quantity in the current time interval has already reached a predefined proportion of the maximum quantity, for example 50%, or if the total quantity reaches or exceeds the maximum quantity.

In particular, the following types of dispensers are preferably used in a system according to the invention.

Preferably, at least one of the dispensers is designed as a liquid dispenser for delivering the substance in liquid form as a non-atomized jet, as a spray jet or in droplet form. Such a liquid dispenser has a liquid store, the volume of which in particular is preferably 20 ml or less. Here, the liquid which contains the active substance is stored before the delivery. The liquid can be stored in the liquid store under pressure or unpressurized. The liquid dispenser also has a delivery opening, through which the liquid is delivered. To effect the delivery, there is provided an actuating handle, i.e. an area on the dispenser to which force is applied. If this actuating handle is actuated, then liquid from the liquid store is delivered to the delivery opening via a pump or via a valve. Preferably, the delivery quantity is always substantially identical. This can easily be implemented, for example, via a pump and its pump chamber volume.

The sensors for detecting the delivery of liquid can be inseparably integrated into the dispenser, for example in the form of a pushbutton on the actuating handle or a flowmeter between liquid store and delivery opening.

Particularly preferred, however, is a configuration in which the liquid dispenser comprises an attachment which includes the sensors for detecting the delivery operation and which can be separated from a main assembly of the dispenser, comprising the liquid store, the delivery channel and the delivery opening, so that after the liquid has been used it can be separated from the latter and can be connected to a new dispenser or the main assembly thereof.

In particular, the attachment can be a device which is attached to the liquid store or to the actuating handle and which has a force or pressure sensor which, during the actuation of the actuating handle, detects the actuation force indirectly.

A second type of dispenser, which is preferably part of a system according to the invention, is a patch dispenser which is provided to deliver patches. A plurality of patches can be kept in the same, each having a store provided with the substance which, when the patch is applied to the skin of a user, rests on the skin.

Here, in particular the following designs are covered by the invention. In a first design, the patch dispenser has a receiving chamber in which the patches are stored. This receiving chamber is delimited by a movable cover element which, in an opened position, permits access to the receiving chamber, so that the user can remove a patch after folding it open. Although the removal itself can be detected by suitable sensors, it is advantageous if the sensors are preferably designed only to detect the opened position. This can be achieved, for example, by a simple force sensor.

Given such a configuration, it is assumed that each opening operation is associated with the removal and use of exactly one patch. If the user removes a plurality of patches, this is not readily detectable by the system. However, provision can be made for the monitoring device, i.e. in particular the smartphone used for this purpose, to permit a user entry with which the user can make it known if they have removed more than one patch or if they have not removed a patch.

A more complex alternative design provides for the patch dispenser to have an actuating handle, by means of which an individual patch can be removed from the patch dispenser. In such a case, the user is thus not given direct access to the receiving chamber of the dispenser but can only remove individual patches therefrom, wherein this in particular involves the actuating handle having to be actuated once for each patch. The detection of the actuation of the actuating handle and/or the delivery of the patch itself can be detected by suitable sensors, in order to transmit this information to the central monitoring device.

A further type of dispenser, which is preferably part of a system according to the invention, is a chewing gum dispenser or a tablet dispenser.

Consistent with the embodiments of the patch dispenser, here, too, the two designs described are possible, that is to say firstly a design in which the dispenser has a receiving chamber in which chewing gum or tablets are stored and which is accessible via a movable cover element and, secondly, a design in which an individual tablet or an individual piece of chewing gum is delivered via an actuating handle. With the exception of the filling and possibly the dimensions of the receiving chamber, such a dispenser can basically be structurally identical to the patch dispenser described.

In the case of chewing gum dispensers and tablet dispensers, however, a further design is considered to be particularly advantageous. In this further design, the chewing gum dispenser or tablet dispenser has a casing into which a packaging means with individual pieces of chewing gum or tablets is inserted. The packaging means can be in particular a blister pack, which has individual compartments for one tablet or one piece of chewing gum each. This packaging means is at least partly withdrawn from the casing as intended for the purpose of dispensing a tablet or a piece of chewing gum.

To detect a delivery operation, such a chewing gum dispenser and tablet dispenser either has a sensor which permits the packaging means to be withdrawn or a sensor is provided which detects the release of a blocking means which counters the withdrawal of the packaging means. Thus, for example, a press stud, the two parts of which must be released in order then to be able to withdraw the packaging means, can be provided at an open end of the casing. The aforementioned press stud can additionally represent a sensor, which makes it possible to detect that the user has opened the press stud, presumably to remove a tablet or a piece of chewing gum.

A fourth type of dispenser preferably provided in the system according to the invention is a dispenser for delivering liquid cartridges. These liquid cartridges themselves are used as a liquid store for a delivery device which, in particular, can be a vaporizer which is designed for the use of interchangeable liquid cartridges. Such a vaporizer is in particular preferably also part of the system according to the invention.

As also already explained in relation to the chewing gum or tablet dispenser and the patch dispenser, provision is also preferably made in this dispenser for delivering liquid cartridges for this to be designed according to one of the designs described there. It therefore preferably has either a receiving chamber, in which the liquid cartridges are stored and which is accessible through a movable cover element, wherein the opening of this cover element is preferably detected by means of a sensor, or it has an actuating handle, which permits an individual delivery of a liquid cartridge, wherein the actuation of the actuating handle or the delivery of the liquid cartridge is detected via a sensor.

The liquid cartridges themselves have a storage container delimited by a wall, which is filled with the substance in liquid form and which has a removal connection, by means of which it is connected to an inlet channel of the aforementioned delivery device, so that the liquid contained can reach a mouthpiece of the delivery device, where, preferably, electrical heating of the liquid for the purpose of the vaporization takes place.

All the aforementioned types of dispensers are distinguished by the fact that they have sensors which detect the delivery of the substance or an action associated with the delivery of the substance. A sensor provided here can preferably be designed very simply, for example in the form of a force or pressure sensor. Furthermore, one of the dispensers for the system has at least one integrated circuit, which is capable of evaluating the sensor and which is additionally designed to communicate with the central monitoring device via the preferably wire-free data interface. This data communication is preferably provided bidirectionally and in particular preferably follows a WLAN or Bluetooth standard. However, a radio standard deviating therefrom is also conceivable and, if appropriate, even unidirectional communication, within the context of which the dispenser does not receive any kind of data but is only designed to transmit. Preferably, the dispensers have an energy source in the form of a battery or a rechargeable battery. However, also possible are dispensers which are designed to harvest energy, that is to say to obtain electrical energy from the action for the purpose of delivering the substance, which energy is then used to produce the radio signal. As already described above, the dispensers also preferably have a store in order to be able to buffer detected delivery or removal processes, in particular when a radio link to the central monitoring device just cannot be established.

### BRIEF DESCRIPTION OF THE DRAWINGS

Further advantages and aspects of the invention can be gathered from the claims and from the following description of preferred exemplary embodiments of the invention, which are explained below by using the figures.

FIG. 1 shows a system according to the invention for delivering nicotine and for monitoring this delivery in an overall illustration.

FIGS. 2 and 3 show patch dispensers, which can be part of the system of FIG. 1.

FIG. 4 shows a liquid dispenser, which can be part of the system of FIG. 1.

FIGS. 5 to 7 show tablet or chewing gum dispensers, which can be part of the system of FIG. 1.

FIGS. 8 and 9 show liquid cartridge dispensers, which can be part of the system of FIG. 1.

### DETAILED DESCRIPTION OF THE EXEMPLARY EMBODIMENTS

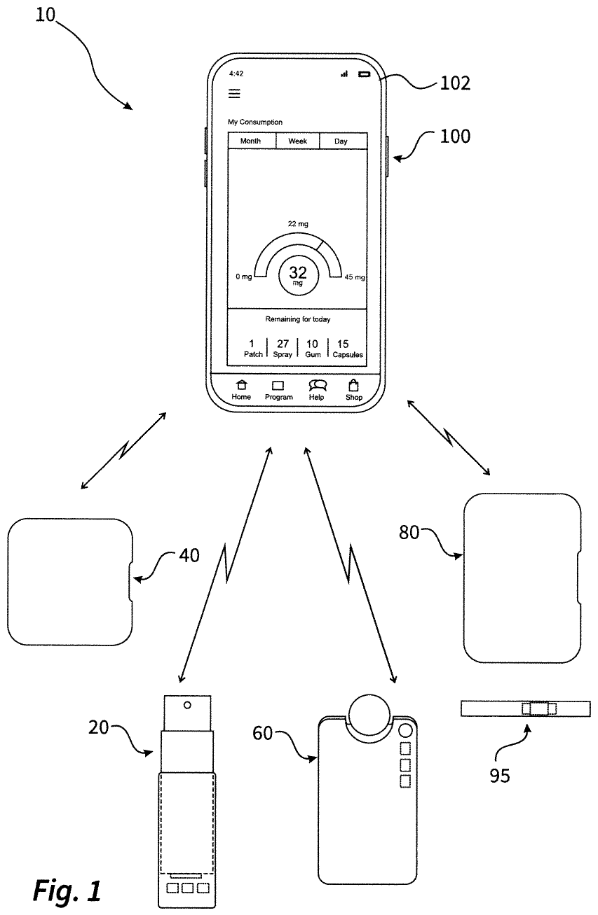

FIG. 1 shows a system 10 according to the invention for delivering nicotine and for monitoring this delivery, in an overall illustration.

The system comprises a central monitoring device 100 which, in the present case, is formed by a smartphone 100. Many people have such a smartphone 100 nowadays, so that this already available device can usually advantageously be used as a central monitoring device. The smartphone 100 has a touch display 102, on which information can be displayed and entries can be made. Installed on the smartphone 100 is a program (app), which provides the monitoring functionality of the system.

The system 10 further comprises a plurality of dispensers 20, 40, 60, 80, which are each designed for the delivery of a medicament or nicotine.

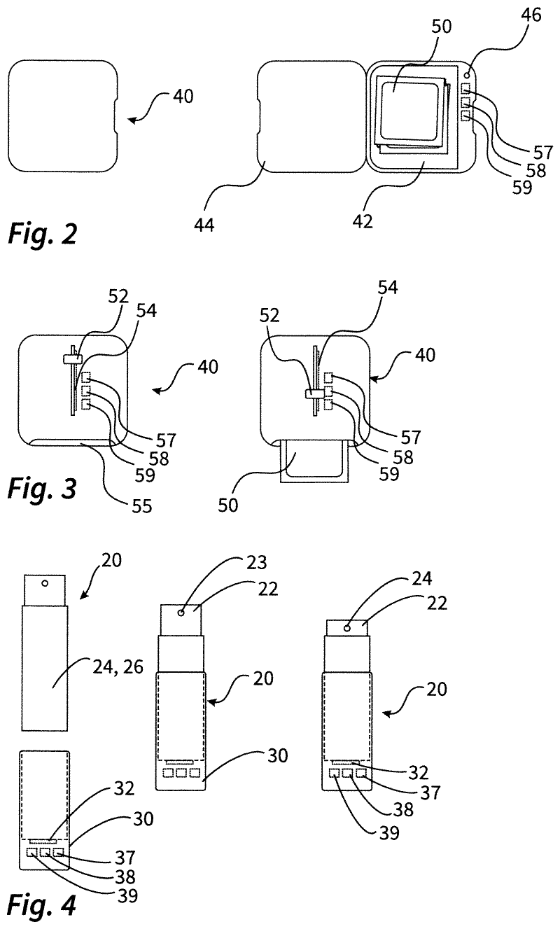

The dispenser 40 is a patch dispenser 40 which, for example, can be configured according to one of the configurations of FIGS. 2 and 3. From the patch dispenser 40 is possible to remove or deliver individual patches 50, which are then applied by the user to their skin, so that they can deliver active substance, such as in particular nicotine, over a relatively long time period.

If a patch is removed or delivered, then this information is transmitted from the patch dispenser 40 to the smartphone 100 via a wire-free radio interface, such as via Bluetooth Low Energy (BLE), and processed by the app there. In the simplest case, the transmission can include only the information relating to the removal or the delivery itself. In addition, however, information, for example relating to the quantity of active substance or to the time of day of the delivery, can also be transmitted. The system 10 in a simple configuration can assume that a patch that is removed or delivered is applied immediately to the skin of the user. However, in another configuration, the app can also permit confirmation via a dialog that the application to the skin has been carried out.

The dispenser 20 is designed as a liquid dispenser 20, which delivers liquid by manual actuation of an actuating handle 22. A liquid dispenser 20 that can be used for this purpose is illustrated in more detail in FIG. 4. During the delivery, the liquid is preferably introduced directly into the human body and, in particular, applied directly to mucosa. In particular, the liquid dispenser can be provided for application of the liquid into eyes, nose or mouth of the user.

The liquid dispenser 20 has a liquid store 24, against which the actuating handle 22 can be pressed down to deliver liquid. The pressing down of the actuating handle 22 and therefore the delivery effected indirectly thereby through a delivery opening on the actuating handle 22 can be detected by appropriate sensors. The information about the delivery, possibly supplemented by additional data such as the delivery quantity and/or the time of day of the delivery, is transmitted to the smartphone 100 and the app there.

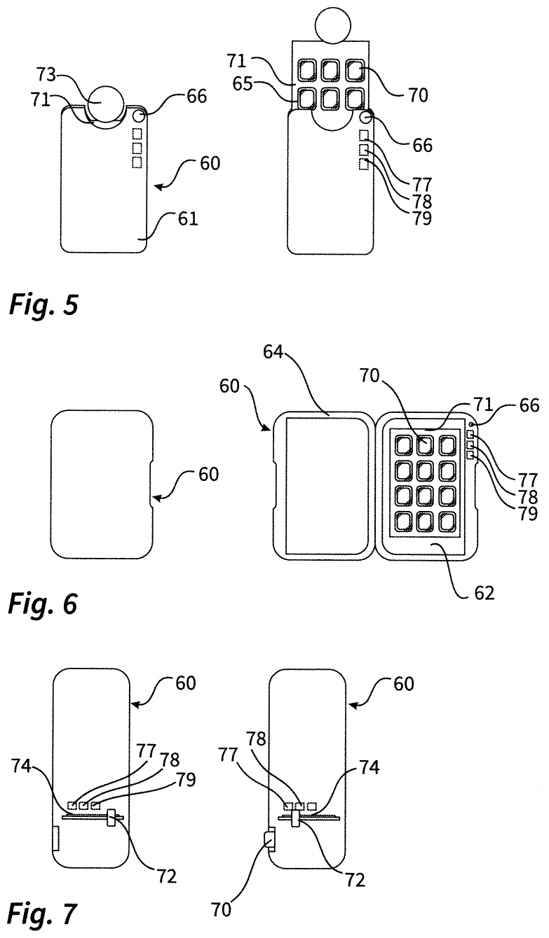

The dispenser 60 is a chewing gum dispenser 60 or tablet dispenser 60 which, for example, can be designed according to one of FIGS. 5 to 7. Individual pieces of chewing gum 70 or tablets 70 can be removed or delivered from the dispenser 60, and can then be swallowed or chewed by the user, so that the active substance contained is ingested by the body.

If a piece of chewing gum or a tablet is removed or delivered, then this information is in turn transmitted from the dispenser 60 to the smartphone 100 via a wire-free radio interface and processed by the app there. Either the app assumes that the use of the chewing gum or the tablet 70 has been made immediately, or the app displays a dialog on the display 102 in order to request confirmation from the user relating to the intake of the chewing gum or the tablet.

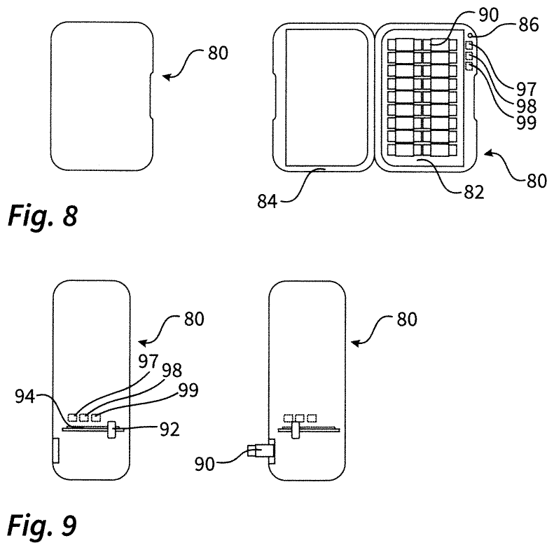

The dispenser 80 is a dispenser for liquid cartridges 90 which, for example, can be designed as illustrated in FIGS. 8 and 9. The liquid cartridges 90, following removal from the dispenser 80, are brought to use as intended by means of separate delivery devices 95, in particular of vaporizer types, wherein the user can breathe in or inhale the in particular vaporized content via such a delivery device.

If a liquid cartridge 90 is removed or delivered from the dispenser 80, then this information is once more transmitted from the dispenser 80 to the smartphone 100 via a wire-free radio interface and processed by the app there. Either the app assumes that the liquid cartridge 90 is inserted into the delivery device 95 immediately and used, or the app requests the time of use via the display 102.

The central monitoring device 100, formed by a smartphone 100 in the present case, thus receives information relating to the delivery of active substance, possibly supplemented by the quantity of active substance actually delivered, from various dispensers 20, 40, 60, 80.

The central monitoring device 100 can store this information separately according to dispenser 20, 40, 60, 80 and then add the respective quantities of active substance to a sum, for example a daily sum, in order to calculate the overall consumption of the medicament or of nicotine. The corresponding value, 32 milligrams at the present time in the exemplary embodiment, is output on the display 102, so that the user is informed hereby. Furthermore, the display indicates that the envisaged maximum quantity per day is around 45 milligrams in the present case, and how many patches 50, pieces of chewing gum 70, liquid cartridges 90 or spray shots by the liquid dispenser are permissible for the remaining 13 milligrams.

In the case of the use for nicotine, the user can thus keep an overview in a very easy form and divide up the residual quantity remaining as far as the maximum quantity in an individually desired way.

Examples of possible dispensers are illustrated and explained in somewhat more detail by using the further figures.

FIG. 2 shows a first variant of a patch dispenser 40 in the closed and opened state. The patch dispenser 40 has a receiving chamber 42, in which a plurality of patches 50 are stored. The receiving chamber can be made accessible from outside by pivoting open a cover element 44, so that a patch 50 can then be removed. The opening of the dispenser 40 is detected via a sensor 46, for example a feeler, and is transmitted to the monitoring device 100 by a processor 57 and a radio module 58, which are fed by a battery 59.

FIG. 3 shows a second variant of a patch dispenser 40. This likewise has a receiving chamber for patches 50. However, this chamber is not immediately accessible. Instead, the dispenser 40 has a delivery slot 55, through which patches 50 can be pushed out individually by means of an actuating handle 52. By means of a sensor 54, for example of a potentiometer type, this can be detected. Once more, the information relating to the delivery is transmitted to the monitoring device 100 via a processor 57 and a radio module 58.

FIG. 4 shows a variant of a liquid dispenser 20. In the manner already described, this has a liquid store 24 against which an actuating handle 22 can be pressed down in order to actuate an internal pump or to open a delivery valve. The liquid can be delivered through a delivery opening 23 in the form of a non-atomized jet or a spray jet or else in the form of individual droplets. Configurations in which a predefined quantity of liquid is delivered are in particular preferred.

Although a conceivable technical design provides for a directly integrated sensor to detect this actuation, it is preferable for the liquid dispenser 20 to have an attachment 30, which is non-essential for the functionality of the delivery itself and which is additionally attached in order to be able to detect the activation. In the present case, the attachment 30 has a tubular section, into which a housing part 26 of the liquid dispenser 20 is inserted. At the bottom of this tubular section, a force sensor 32 is provided which, when force is applied to the handle 22, detects the corresponding force and in this way is able to detect whether an actuation and supposedly a delivery has taken place. If an actuation and a delivery operation have taken place, this is transmitted to the monitoring device 100 by the processor 37 and the radio module 38.

FIGS. 5 to 7 show different variants of a chewing gum dispenser 60. In the configuration of FIG. 5, this has a casing 61, for example of artificial leather, into which a blister pack 71 having a large number of pieces of chewing gum 70 is inserted. The blister pack 71 can be drawn out of the casing 61 via a pulling element 73, but only when a press stud 66 has previously been released. This press stud 66 is simultaneously a sensor, so that the processor 77 can detect that the press stud has been released. This is assessed as a signal that the user has drawn out the blister pack 71 and removed and used a piece of chewing gum 70. This information is transmitted to the monitoring device by means of the radio module 78.

The variants of the chewing gum dispenser of FIGS. 6 and 7 correspond substantially to the patch dispensers of FIGS. 2 and 3. As described in relation to FIG. 2, the configuration of FIG. 6 represents a dispenser with a receiving chamber 62 in which, in this case, individual pieces of chewing gum are stored, possibly in a blister pack 71. Opening of the cover element 64 is detected by means of a sensor 66 and reported to the monitoring device 100 via the radio module 78. The configuration of FIG. 7, corresponding to FIG. 3, is a dispenser for individual delivery, in this case of chewing gum 70, which can be delivered individually by means of an actuating handle 72 in order to be consumed by the user. The actuation of the actuating handle 72 is detected via a suitable sensor 74, and the associated delivery is transmitted to the monitoring device 100.

FIGS. 8 and 9 show dispensers 80 for liquid cartridges 90. As already described above, these are cartridges which are used in delivery devices such as vaporizers, in order to be able to deliver the liquid contained therein.

The dispensers of FIGS. 8 and 9 correspond substantially to the patch dispensers of FIGS. 2 and 3. The dispenser of FIG. 8 permits access to a receiving chamber 82, in which the cartridges are stored and from which they can be removed, wherein the sensor 86 detects the opening of the cover element 84, so that this can be transmitted to the monitoring device 100 by the processor 97 and the radio module 98. The dispenser of FIG. 9 once again permits the individual delivery of the cartridges 90, wherein the delivery is detected by a sensor 94 on the actuating handle 92 and is reported to the monitoring device 100.

1. A system for monitoring the intake of substances including pharmaceutical substances or nicotine, comprising:
at least two dispensers for delivering the same substance, the at least two dispensers being configured to deliver the same substance in a different application form; and
a monitoring device connected to the at least two dispensers by a data link;
wherein the at least two dispensers are configured to detect an action associated with the delivery of the substance and, in reaction hereto, to transmit data relating to the delivery of the substance to the monitoring device; and
wherein the monitoring device is configured to receive the data relating to the delivery of the substance from the at least two dispensers and to calculate a total quantity of the delivered substance.

2. The system as claimed in claim 1, wherein the substance contains nicotine.

3. The system as claimed in claim 1, wherein the monitoring device has an indicating device and is configured to display the total quantity of the delivered substance on the indicating device.

4. The system as claimed in claim 1, wherein the monitoring device is configured to calculate the total quantity of the delivered substance over a limited time period.

5. The system as claimed in claim 1, further including at least one of the following:
the monitoring device is configured to compare the total quantity of the delivered substance with a predefined maximum quantity and to output a warning message if the total quantity is reached and/or exceeded; and/or
the monitoring device is configured to compare the total quantity of the delivered substance with an envisaged maximum quantity and to calculate what delivery quantity is still available on the current day, in the current week or in the current month.

6. The system as claimed in claim 1, wherein at least one of the at least two dispensers is configured as a liquid dispenser for delivering the substance in liquid form as a non-atomized jet, as a spray jet or in droplet form.

7. The system as claimed in claim 1, wherein at least one of the at least two dispensers is configured as a patch dispenser for delivering patches.

8. The system as claimed in claim 1, wherein at least one of the at least two dispensers is configured as a chewing gum dispenser or a tablet dispenser.

9. The system as claimed in claim 1, wherein at least one of the at least two dispensers is configured to deliver liquid cartridges, and the liquid cartridges are couplable to a delivery device.

10. The system as claimed in claim 1, wherein at least one of the at least two dispensers is configured as a patch dispenser and at least one of the at least two dispensers is configured as a liquid dispenser.

11. The system as claimed in claim 1, wherein at least one of the at least two dispensers is configured as a patch dispenser and at least one of the at least two dispensers is configured as a chewing gum dispenser or a tablet dispenser.

12. The system as claimed in claim 1, wherein at least one of the at least two dispensers is configured as a patch dispenser and at least one of the at least two dispensers is configured as a dispenser for delivering liquid cartridges.

13. The system as claimed in claim 1, wherein at least one of the at least two dispensers is configured as a liquid dispenser and at least one of the at least two dispensers is configured as a chewing gum dispenser or a tablet dispenser.

14. The system as claimed in claim 1, wherein at least one of the at least two dispensers is configured as a liquid dispenser and at least one of the at least two dispensers is configured as a dispenser for delivering liquid cartridges.

15. The system as claimed in claim 1, wherein at least one of the at least two dispensers is configured as a chewing gum dispenser or a tablet dispenser and at least one of the at least two dispensers is configured as a dispenser for delivering liquid cartridges.

16. The system as claimed in claim 1, further including at least one of the following:
the data link is a WLAN or Bluetooth data link or a GSM, 3G, 4G or 5G mobile radio link; and/or
the monitoring device and at least one of the at least two dispensers are configured for a coupling operation wherein an unambiguous code on the dispenser is read by a smartphone; and/or
the monitoring device comprises a computer.

17. A method for monitoring the intake of a substance from a plurality of dispensers in a different application form, the method comprising:
detecting handling, with sensors, of at least two dispensers configured to deliver a substance in different application forms; and
sending data, via the at least two dispensers, relating to the delivery of the substance to a monitoring device in reaction thereto; and
receiving, with the monitoring device, the data and on the basis of the received data,
calculating a total quantity of the substance delivered by the at least two dispensers.

18. The method as claimed in claim 17, further including detecting, with
at least one of the at least two dispensers a handling of the dispenser, the handling being used to open a receiving chamber, and sending data to the monitoring device as a reaction thereto.

19. The method as claimed in claim 17, further including detecting, with at least one of the at least two dispensers, a handling of the dispenser which causes the delivery of the substance, and sending data to the monitoring device as a reaction thereto.

20. The system as claimed in claim 6, further including at least one of the following:
the at least one liquid dispenser has an actuating handle and is configured to detect an actuation of the actuating handle; and/or
the at least one liquid dispenser has an attachment detachably coupled to a housing part of the liquid dispenser and is configured to detect the handling of the liquid dispenser; and/or
the at least one liquid dispenser has a liquid store containing the substance; and/or
the at least one liquid dispenser is configured as a pump dispenser.

21. The system as claimed in claim 7, further including at least one of the following:
the patch dispenser has a receiving chamber for receiving the patches, a movable cover element, the movable cover element in an opened position permitting access to the receiving chamber and the removal of a patch, the patch dispenser further including a sensor for detecting the opened position; and/or
the patch dispenser has an actuating handle permitting removal of an individual patch from the patch dispenser, and a sensor for detecting actuation of the actuating handle; and/or
the patch dispenser is filled with a plurality of patches, each patch having a store containing the substance, the substance, when the patch is applied to the skin of a user, resting on the skin.

22. The system as claimed in claim 8, further including at least one of the following:
the chewing gum dispenser or the tablet dispenser has a casing containing packaging means with individual pieces of chewing gum or tablets, wherein the packaging means is drawable out of the casing to permit removal of a piece of chewing gum or a tablet, and a pulling out of the packaging means, or release of a block in order to be able to pull out the packaging means, is detectable by a sensor; and/or
the chewing gum dispenser or the tablet dispenser has a receiving chamber for receiving a piece of chewing gum or tablets and a movable cover element, the movable cover element in an opened position permitting access to the receiving chamber and removal of a piece of chewing gum or a tablet, the chewing gum dispenser or tablet dispenser having a sensor for detecting the opened position; and/or
the chewing gum dispenser or the tablet dispenser has an actuating handle, the actuating handle permitting an individual piece of chewing gum or an individual tablet to be removed from the chewing gum dispenser or the tablet dispenser, the chewing gum dispenser or the tablet dispenser having a sensor for detecting actuation of the actuating handle; and/or
the chewing gum dispenser or the tablet dispenser is filled with a plurality of pieces of chewing gum or tablets, each of the pieces of chewing gum or each of the tablets containing the substance.

23. The system as claimed in claim 9, further including at least one of the following:
the dispenser has a receiving chamber for receiving the liquid cartridges and a movable cover element, the movable cover element, in an opened position, permitting access to the receiving chamber and removal of a liquid cartridge, the dispenser further including a sensor for detecting the opened position; and/or
the dispenser has an actuating handle permitting removal of an individual liquid cartridge from the dispenser, and a sensor for detecting actuation of the actuating handle.

24. The method as claimed in claim 19, further including at least one of the following steps:
detecting application of a force and/or a displacement of an actuating handle with a sensor of the dispenser; and/or
carrying out the delivery in liquid form as a non-atomized jet, as a spray jet or as droplets; or
carrying out the delivery in the form of a patch, a piece of chewing gum or a tablet, to be ingested by a user following the delivery; or
carrying out the delivery in the form of a liquid cartridge inserted into an applicator following the delivery.
